(19) United States
(12) Patent Application Publication
(10) Pub. No.: US 2008/0097485 A1
(43) Pub. Date: Apr. 24, 2008

(54) Device for wound suturing and hemostasis in the thoracic and the abdominal wall mainly in endoscopic operations

(76) Inventors: Vladimir Shpaichler, Jerusalem (IL); Evgeny Shpaichler, Jerusalem (IL)

Correspondence Address: FAY SHARPE LLP, 1100 SUPERIOR AVENUE, SEVENTH FLOOR, CLEVELAND, OH, 44114, US

(21) Appl. No.: 11/975,219

(22) Filed: Oct. 18, 2007

Related U.S. Application Data
(63) Continuation-in-part of application No. PCT/IL2006/000443, filed on Apr. 6, 2006.

(30) Foreign Application Priority Data
Apr. 19, 2005 (IL) 168129

(51) Int. Cl. A61B 17/04

(52) U.S. Cl. 606/148

(57) ABSTRACT

The aim of the device is to achieve direct and supervised complete closure of the Port Site wound. The following advantages will be procured simultaneously: 1. Preservation of the initial shape of the wound channel. 2. Preservation of the stable layer structure of the Port Site wound during the suturing. 3. Precise, supervised, semi-automatic insertion of the thread-guiding members.

### CROSS REFERENCE TO RELATED APPLICATION

This application is a continuation-in-part application of PCT Appl. No. PCT/IL2006/000443 (Int. Publ. No. WO 2006/111955) filed Apr. 6, 2006, titled “Device for Wound Suturing and Hemostasis in the Thoracic and the Abdominal Wall Mainly in Endoscopic Operations”. The aforementioned PCT application claimed priority from Israel Pat. App. 168129, filed Apr. 19, 2005.

### FIELD OF THE INVENTION

The present invention relates to the field of medicine and veterinary medicine and, more particularly, to devices for wound suturing and hemostasis in endoscopic, mainly, laparoscopic and thoracoscopic, operations. The invention may also be used for suturing wounds of any etiology similar to those formed during the endoscopic operations.

### STATE OF THE ART

As it is known, an endoscopic operation comprises the following steps:

insertion of a port through the abdominal or the thoracic wall, into the abdominal or the thoracic cavity, respectively, with the resultant formation of a wound and wound channel,

introduction of a surgical or a diagnostic instrument into the abdominal or the thoracic cavity through the port channel,

performing the operation or the diagnostic procedure with the help of the instrument,

removing the instrument,

removing the port,

suturing the wound in the abdominal or the thoracic wall.

It should be mentioned that once the port is removed the wound becomes an unstable layer structure and the wound channel changes its shape.

At present, two methods of wound suturing upon the removal of the port are known.

According to one of them, the wound is sutured manually with the help of surgical thread-guiding members, for example clamp needles, and without any special devices. This method is highly traumatizing and labor-consuming as the selection of the right directions of the thread-guiding members inserted through the abdominal or the thoracic wall into the pre-specified points of the body requires quite a considerable amount of time.

Moreover, upon the removal of the port the wound becomes an unstable layer structure. Substantial disarrangement of approximation of the layers of the abdominal or the thoracic wall takes place during the wound suturing. The disarrangement of approximation is also contributed to by the circumstance that a strictly directed suture as required by surgical standards is impossible to achieve manually. In its turn, the disarrangement of approximation leads to formation of inter-tissue cavities causing various post-surgery complications such as bleeding, suppuration, and hernias.

The other method involves the use of special devices which diminish the traumatizing effect of wound suturing and the amount of labor required.

In practice, a device used until recently for wound suturing in laparoscopic operations comprised of a tubular body with a suturing unit built therein. It included two needles with threads positioned parallel to the longitudinal axis of the tubular body. When such device is inserted into a body cavity and the suturing unit is advanced out of the tubular body the needles move apart. The tubular body together with the suturing unit is then drawn upwards and the abdominal wall is pierced by the parallel needles. To remove the device from the abdominal cavity, the tubular body is inserted into the cavity again and the suturing unit is returned into the tubular body. The use of this device reduces the traumatizing effect of suturing but layer approximation remains significantly disarranged because of the parallel motion of the threads through the thick sutured tissue.

Another device for wound suturing in laparoscopic operations is known (Catalogue of “Inlet Medical, Inc.”, 2002, Carter—Thomason Instruments, Art. No CTI-SE). The device comprises a cone-shaped member having a pair of guides for two thread-grasping members, or graspers, functioning jointly during the wound suturing. The guides are oriented in such a manner that when the cone-shaped member is in the wound channel they determine the trajectories of motion of the thread-grasping members. The thread-grasping members are inserted through the abdominal wall into zones containing preset points.

The cone-shaped member is inserted with its apex into the wound channel when the port is removed. The graspers are then inserted into the preset points through the guides in the cone-shaped member.

The use of this device reduces the traumatizing effect during wound suturing but even in this case the wound remains an unstable layer structure, and the approximation of layers of the abdominal wall is disarranged during wound suturing, as described above. This is accounted for, primarily, by the circumstance that the wound surface only partially contacts that of the cone-shaped member.

Besides, after the port is removed, the search for the wound channel with the cone apex is often difficult and may cause a change in the wound configuration and a further disarrangement of its layer structure.

Finally, the necessity of searching for the wound channel while using the above devices makes wound suturing after an operation much more difficult.

### SUMMARY OF THE INVENTION

The object of the present invention is, therefore, the task of developing a device for wound suturing and hemostasis in endoscopic operations that would allow to preserve the initial shape of the wound channel and the stable layer structure of the wound at the moment of suturing thereby diminishing the probability of post-surgery complications. The object of the present invention is further to provide a device that will ensure the initially correct directions of motion of thread-guiding members inserted through the abdominal or the thoracic wall into pre-specified points of the body thereby diminishing the traumatizing effect of wound suturing and reducing labor and time expenditures connected with suturing.

The device of the present invention comprises a body having a tubular wall with an outer cross-sectional perimeter matching the cross-sectional perimeter of a pre-specified wound channel. The length of the tubular wall exceeds the preset length of the wound channel. There is at least one pair of guides on the body for two thread-guiding members. Without intending to limit the nature of these members, these members may be needles. These thread guiding members function jointly during wound suturing and hemostasis. The guides are fixed on the body and oriented so as to ensure that the trajectories of motion of the thread-guiding members that guide the threads are lead into the pre-specified points of the body through the abdominal or the thoracic wall.

The present invention will be understood more fully from the detailed description given herein below and from the accompanying drawings of the preferred embodiment of the invention which, however, should not be construed as exhaustive to the invention but are for explanation and understanding only.

### BRIEF DESCRIPTION OF THE DRAWINGS

The present invention is illustrated by drawings wherein:

FIG. 1 shows a general view of the device of the present invention;

FIG. 2 shows a general view of the device in FIG. 1 in its operating position;

FIG. 3 is a view along arrow A in FIG. 2;

FIG. 4 shows a general view of the device of the present invention in its operating position used for another purpose; and

FIG. 5 is a view along arrow B in FIG. 4.

### DETAILED DESCRIPTION OF THE PREFERRED EMBODIMENTS OF THE INVENTION

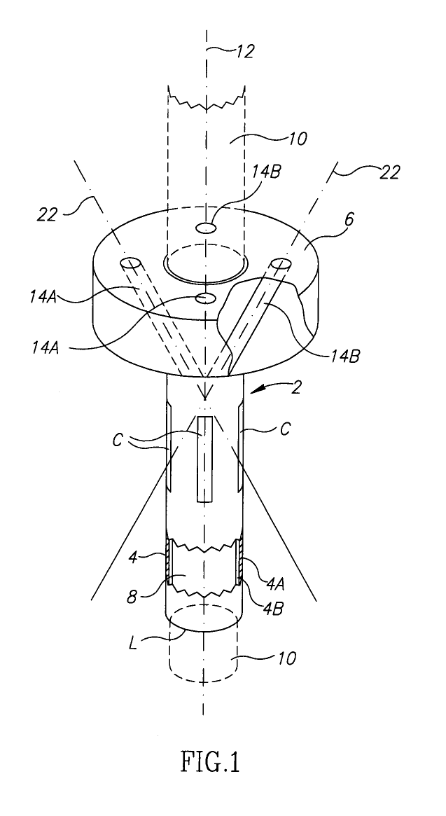

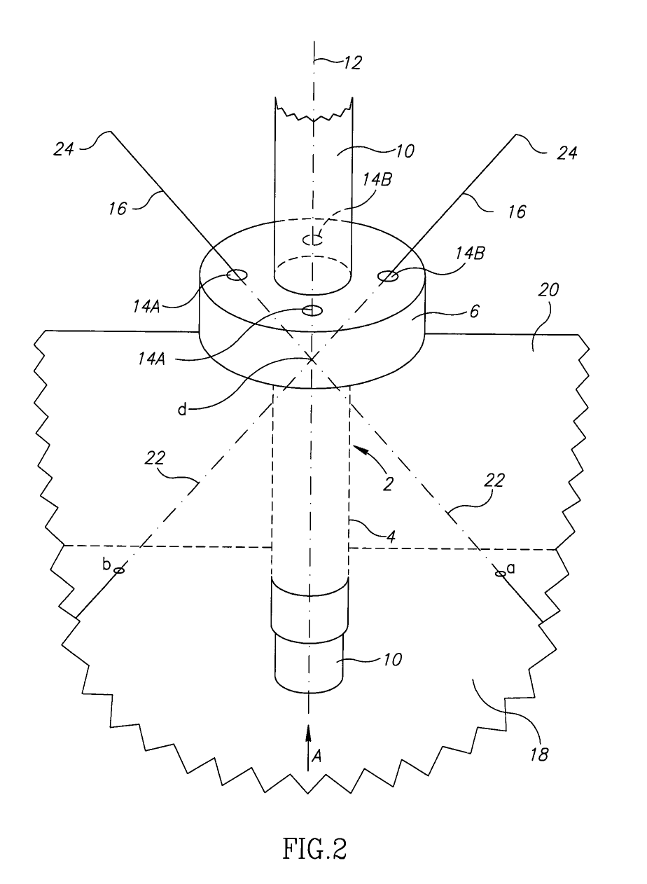

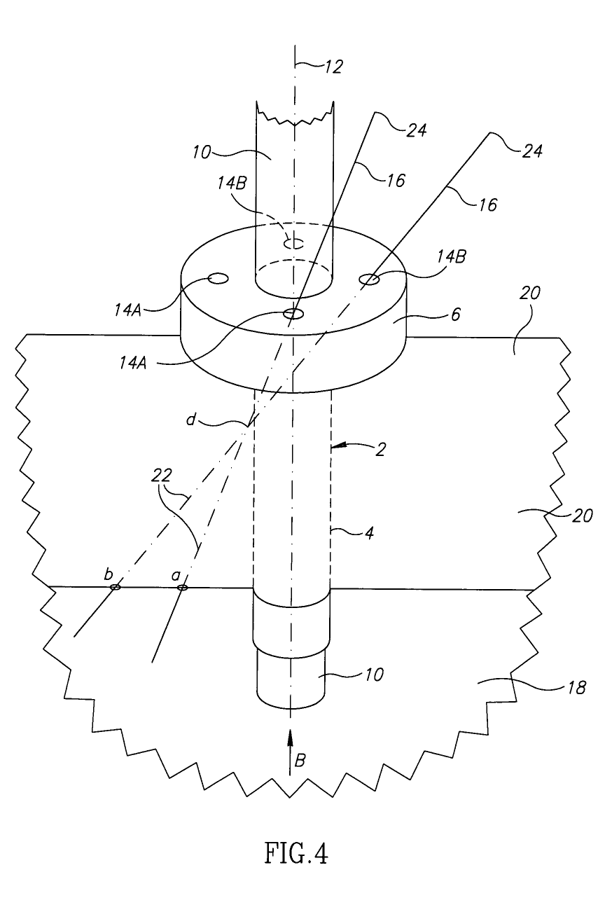

The proposed device for wound suturing and hemostasis is used after endoscopic operations wherein a port, also at times referred to herein as an endoscopic access port, is employed. The device (FIG. 1)_comprises body 2 having a cylindrical tubular wall 4 equipped with a cap 6 on one end thereof. Tubular wall 4 is intended to preserve the initial shape of wound channel 8 (FIGS. 2,3) during the entire operation. Embodiments of the present invention are possible wherein the tubular wall has other than a cylindrical shape. Outer side 4a of wall 4 in its shape and size (length “L” of the outer cross-sectional perimeter of outer side 4a of wall 4) matches the preset shape and size of wound channel 8. The term “size of wound channel” denotes the cross-sectional perimeter of the wound channel wall. “Size matching” means the equivalence of sizes with acceptable deviations small enough to allow the fulfillment of the object of the present invention. Matching is easy to determine experimentally. In another embodiment, the cross-sectional shape of our device may be different from the cross-sectional shape of the wound channel. For example it may be star-shaped. In such a case the shape of the body wall will be chosen in order to preserve the stable layer structure of the wound. Inner side 4b of wall 4 has a diameter making it possible for port 10 to be moved inside wall 4 along its longitudinal axis 12. In cap 6, there is provided paired guides 14a and 14b formed in the shape of slots. These guides are intended to guide thread-guiding members 16, the latter typically, but without intending to limit the invention, being needles. Guides 14a, 14b of each pair are positioned symmetrically relative to axis 12. Guides 14a, 14b are oriented so that when port 10 is in its operating position they ensure such trajectories of motion of thread-guiding members 16 (FIGS. 2, 4) that the threads guided by them are lead into pre-specified points “a” and “b” in abdominal or thoracic cavity 18 through abdominal or thoracic wall 20, respectively. By “operating position” of the port such position is meant whereby tubular wall 4 is inserted into the wound channel throughout the length of the latter. The number of guides 14a, 14b and their orientation are determined by the specific purpose of the device (wound suturing, hemostasis or both). The areas of tubular wall 4 located in zone “c” of trajectories 22 of motion of thread-guiding members 16 are made of a material that may be pierced through by thread-guiding members 16, for example, of silicon. Such an arrangement does not require modification of the design of existing ports.

The proposed device is operated as follows.

Before the beginning of an endoscopic operation the port 10 is inserted into tubular wall 4 of body 2 and is fixed therein by any of several known methods, for example, by means of a threaded connection (not shown for considerations of simplicity). Tubular wall 4 together with port 10 is then inserted through abdominal or thoracic wall 20 into abdominal or thoracic cavity 18, respectively. Thereby a wound—wound channel 8—is formed in abdominal or thoracic wall 20. After that, a surgical or a diagnostic instrument (not shown) is inserted into abdominal or thoracic cavity 18 through the inner cavity of port 10, a surgical or a diagnostic manipulation is performed, and the instrument is removed. Then port 10 is detached from body 2 and removed therefrom whereas tubular wall 4 stays in wound channel 8 providing for a reliable preservation of the initial shape of wound channel 8 and for the stability of the wound layer structure.

Another embodiment of the present invention is possible wherein the device is used separately from the port. In this case the port is inserted into abdominal or thoracic cavity 18 without the proposed device. Before the expected removal of the port from the wound channel a guide (not shown) is passed through the port whereupon the latter is removed. The guide staying in the wound channel makes it possible to preserve the stable layer structure of the wound. The device of the present invention is then inserted into the wound along the guide. This arrangement, too, does not involve the modification of the design of existing ports, and, moreover, it is more universal as it allows the same device to be employed in different wound channels with comparable cross-sectional sizes.

Wound suturing with the help of the proposed device is performed as follows.

First thread-guiding member 16 together with (suturing) thread 24 is moved along guide 14a ensuring trajectory 22, to be inserted into preset point “a” of abdominal or thoracic cavity 18, and successively pierces through zone “c” of tubular wall 4, and abdominal or thoracic wall 20. Along guide 14b, second thread-guiding member 16 with graspers (not shown) is inserted into point “b” in a similar manner, the graspers grasping thread 24 to guide it out of the abdominal or thoracic cavity along the same trajectory. Then the device for wound suturing is removed from wound channel 8 and thread 24 is tied forming a suture. A second suture may be made with the help of a second pair of guides 14, when necessary.

Depending on the purpose of suturing (tightening the wound, suppressing bleeding in the wound area, fixing the tissues in the wound area), the suture may cut the central axis of the wound channel or lie beyond it.

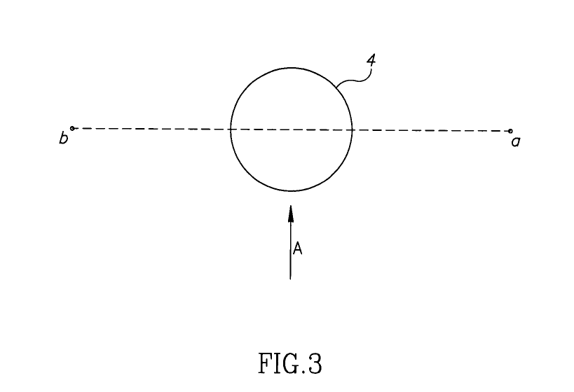

In the former instance, guides 14 are oriented so that point “d” of intersection of trajectories 22 lies on longitudinal axis 12 (FIGS. 2, 3). Such positioning of the suture is optimal for tightening the wound itself.

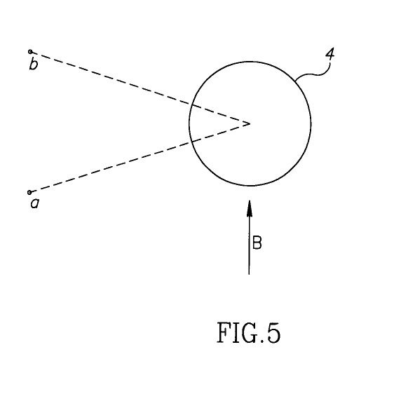

In the latter instance, guides 14 are oriented so that point “d” of intersection of trajectories 22 lies beyond longitudinal axis 12 (FIGS. 4, 5). Such suture is used, for example, to suppress marginal bleeding.

In one of the embodiments of the present invention (not shown in the figures), port 10 itself may be used in the capacity of the body, or its part, of the proposed device. In this case, port 10 itself, or the mentioned additional part of the body, has guides 14 and the zones “c” may be made of a material to be pierced through by thread-guiding members 16. This arrangement, however, may require modifications to the design of the port.

In this arrangement, when the surgical or the diagnostic instrument is removed from cavity 18, port 10 stays in wound channel 8 preserving the initial shape of wound channel 8 and the stable layer structure of the wound. Suturing is performed as described above.

In embodiments where port 10 itself, or one of its parts, functions as body 2 (or part thereof) of the device of the present invention as discussed above, particularly when it serves as the tubular-shaped wall portion of body 2, guides 14 may be positioned in body 2 and oriented by any of many different ways.

The guides may be positioned in a guide containing member (not shown) which may be fixably or removably attached to the port. The guides are fixably oriented within the guide containing member. Without intending to limit the form and types of guide containing members, cap 6 of FIG. 1 may be considered to be an example of a guide containing member.

The guide containing member (not shown) may be made of a plastic or polymeric material integrally molded to the access port, the latter typically made of plastic. Alternatively, the guide containing member may be made from a separate piece fixed to the port by any of many different types of attachment means known to those skilled in the art, such as screws, clips, or elastic means. These attachments are exemplary only and are not intended to be limiting. When the guide containing member is attached to the tubular-shaped wall structure of the port, it may be attached either entirely around the tubular-shaped wall structure of the port or only on one side of the port's wall structure.

When screws, clips, elastomeric means, etc. are used to join a guide containing member to the body, the separate guide containing member may function as a universal guide containing member irrespective of the diameters and/or dimensions of the port's tubular-shaped wall structure.

In some embodiments, the guide containing member may be a removable sleeve or sheath-like member. Typically, the sleeve would be open at both ends and removably placable on the port so that the guides, integrally formed within the sleeve, are substantially adjacent to the tubular-shaped wall of the port.

The sleeve or sheath-like member may be made wholly, or partially, of one or more elastomeric materials or one or more plastic materials.

In some embodiments, the sleeve or sheath-like member may be split along a groove allowing it to expand and be placed on the tubular-shaped portion of ports having different diameters and/or dimensions. The sleeve or sheath-like members discussed above may be considered to be universal guide containing members, suitable for use with a range of ports having different diameters and/or dimensions.

When the attachment structure is made of plastic or elastomeric materials, the structure may be molded to contain as many guides 14 as required. Thread-guide members 16 are insertable into the guides positioned in the guide containing members.

In another embodiment of the present invention (not shown in the figures), outer side 4a of wall 4 may have a shape other than the preset shape of the wound channel. Its cross section may be shaped as an ellipse, a triangle, etc. In this case it is sensible to insert the proposed device into the wound channel with the help of the guide.

In the Figures provided and discussed above, the thread-guiding members have trajectories which intersect at a point. It should be understood that in other embodiments the thread-guiding members need not intersect at a point, but one trajectory may be projected so that its projection intersects another trajectory. However, as noted above, all that is required of guides 14a and 14b in the present invention is that guides 14a and 14b allow for the trajectories 22 of thread-guiding members 16 to pass through pre-specified points in the body cavity.

The use of the device of the present invention allows reliable preservation of the initial shape of the wound channel and of the stable layer structure of the wound, as well as the possibility of making a strictly directed suture. This considerably reduces the disarrangement of approximation of layers of the abdominal or thoracic wall, and, hence, diminishes the risk of post-surgical complications such as bleeding, suppuration and hernias. Additionally, the use of the proposed device eliminates the time-consuming step of searching for the wound channel after the port is removed. This facilitates considerably wound suturing after an operation. The device of the present invention may also be used to fix various tissues and materials inside the abdominal, the thoracic or other cavity.

Although the invention has been described and illustrated with a certain degree of particularity, it is understood that the present disclosure has only been made by way of example, and that various modifications thereof may be resorted to by those skilled in the art without departing from the spirit and scope of the invention, as hereinafter claimed.

What is claimed is:

1. A device for wound suturing and hemostasis for use in endoscopic procedures, wherein a port for use with said device is inserted into a wound via a wound channel, said device comprising:
a body including a wall, the wall having a tubular shape and a length exceeding the length of the wound channel, said tubular-shaped wall maintaining the initial shape of the wound channel and the stable layer structure of the wound from which the wound channel emanates when said device is placed into the wound channel;
at least two thread-guiding members for guiding at least one suture thread attached thereto, said members to function in conjunction with each other to effect wound suturing and hemostasis; and
at least one pair of guides positioned on a portion of said body external to the hollow of said tubular-shaped wall,
wherein said guides are oriented so as to ensure that the trajectories of motion of the at least two thread-guiding members are lead to pre-specified points in a body cavity after passing through a body cavity wall.

2. The device according to claim 1, wherein the perimeter of the outer side of the tubular-shaped wall is essentially identical to the perimeter of the wall of the wound channel.

3. The device according to claim 2, wherein the shape of the outer side of the tubular-shaped wall is essentially identical to the shape of the wound channel.

4. The device according to claim 3, wherein the tubular-shaped wall is formed in the shape of a hollow cylinder able sized to circumscribe the port so as to enable the port to be moved along the tubular shaped wall in the direction of the longitudinal axis of the hollow cylinder.

5. The device according to claim 1, wherein a part of the port forms said tubular-shaped wall of said body.

6. The device according to claim 5, wherein said guides are formed and fixedly oriented in a sheath formed of elastomeric material, said sheath open at both its ends and positioned on said port.

7. The device according to claim 5, wherein said guides are formed and fixedly oriented in a plastic guide containing member fixably attached to said port.

8. The device according to claim 5, wherein said guides are formed and fixedly oriented in a plastic guide containing member removably attached to said port.

9. The device according to claim 1, wherein zones of said tubular-shaped wall of said body are formed of a material which can be pierced by said thread-guiding members, said zones lying on the trajectories of motion of the thread-guiding members.

10. The device according to claim 1, wherein said guides are oriented so as to ensure an intersection of the trajectories of motion of the thread-guiding members with the intersection point of the trajectories lying on the longitudinal axis of said tubular wall.

11. The device according to claim 1, wherein said guides are oriented so as to ensure an intersection of the trajectories of motion of the thread-guiding members with the intersection point of the trajectories lying off the longitudinal axis of the tubular wall.

12. The device according to claim 1, wherein said guides are oriented so that the trajectories of motion of the thread-guiding members, when projected, intersect.

13. The device according to claim 1, wherein said thread-guiding members are needles.

14. The device according to claim 1, wherein said device further includes a cap affixed to an end of said tubular-shaped wall of said body.

15. The device according to claim 14, wherein said guides are positioned in said cap.

16. The device according to claim 15, wherein zones of said tubular-shaped wall of said body are formed of a material which can be pierced by said thread-guiding members, said zones lying on the trajectories of motion of the thread-guiding members.

17. The device according to claim 15, wherein said guides are oriented so as to ensure an intersection of the trajectories of motion of the thread-guiding members with the intersection point of the trajectories lying on the longitudinal axis of said tubular wall.

18. The device according to claim 15, wherein said guides are oriented so as to ensure an intersection of the trajectories of motion of the thread-guiding members with the intersection point of the trajectories lying off the longitudinal axis of the tubular wall.

19. The device according to claim 1 wherein the body cavity is the abdominal or thoracic cavity and the body cavity wall is the abdominal or thoracic cavity wall.

20. A device for wound suturing and hemostasis for use in endoscopic procedures, said device comprising:
a body including an endoscopic access port, said port having a tubular shape and a length exceeding the length of a wound channel into which it is inserted, said port serving to maintain the initial shape of the wound channel and the stable layer structure of the wound from which the wound channel emanates when said port is placed into the wound channel;
at least two thread-guiding members for guiding at least one suture thread attached thereto, said members to function in conjunction with each other to effect wound suturing and hemostasis; and
at least one pair of guides positioned on a portion of said body and external to the hollow of said tubular-shaped port,
wherein said guides are oriented so as to ensure that the trajectories of motion of the at least two thread-guiding members are lead to pre-specified points in a body cavity after passing through a body cavity wall.

21. The device according to claim 20, wherein said guides are formed and fixedly oriented in a sheath formed of elastomeric material, said sheath open at both its ends and positioned on said port.

22. The device according to claim 20, wherein said guides are formed and fixedly oriented in a plastic guide containing member fixably attached to said port.

23. The device according to claim 20, wherein said guides are formed and fixedly oriented in a plastic guide containing member removably attached to said port.
